(19) United States
(12) Patent Application Publication
(10) Pub. No.: US 2024/0335190 A1
(43) Pub. Date: Oct. 10, 2024

(54) ENDOSCOPIC METHOD AND DEVICE FOR SEALING A GASTROINTESTINAL DEFECT

(71) Applicant: GIFTEDNESS AND CREATIVITY COMPANY, Safat (KW)

(72) Inventor: SULAIMAN M M ALMAZEEDI, SAFAT (KW)

(21) Appl. No.: 18/131,176

(22) Filed: Apr. 5, 2023

(51) Int. Cl. A61B 17/00

(52) U.S. Cl. CPC A61B 17/0057; A61B 2017/00597; A61B 2017/00632; A61B 2017/00641; A61B 2017/00818

(57) ABSTRACT

A system and method for sealing a gastrointestinal defect includes using an endoscope, colonoscope, or gastroscope for the deployment of a gastrointestinal plug into the area of the gastrointestinal defect. Once optimally positioned, the gastrointestinal plug expands on either side, or both sides, of the opening associated with the defect, effectively sealing off the defect.

### BACKGROUND

### 1. Field

The present disclosure relates to gastrointestinal procedures and devices, and particularly to an endoscopic method and device for sealing a bleeding gastrointestinal defect, such as an ulcer, a gastrointestinal perforation, a post-bariatric surgery leak, a gastrointestinal fistula or the like.

### 2. Description of the Related Art

The gastrointestinal system begins in the mouth and ends in the anus, and includes the stomach, small intestine, large intestine, and the colon. There are many conditions that can cause perforations and leaks in the gastrointestinal tract, and these most commonly occur after surgical procedures. One of the most common surgical procedures performed around the world today is a laparoscopic sleeve gastrectomy. The surgery involves removing around 80% of the stomach in order for the patient to lose weight and treat obesity. Even though the surgical procedure is very common, around 1-5% of all cases developed a leak along a staple line. When this occurs the treatment is quite difficult with many centers around the world employing different techniques with limited success.

The most common technique performed today is placing a stent across the leak defect in hopes that this will lead to eventual closure. This, however, can be associated with very severe side effects experienced by the patient and can require no less than six weeks and two stents for recovery. During this time, the patient cannot eat and must be fed through a tube.

A second common cause of perforations and leaks in the gastrointestinal tract occurs when surgery is performed on the colon, especially for cancer, which can sometimes bring on complications from the procedure due to leaks along the staple line after a resection. These leaks are also very difficult to manage and most of the time end up with the patients requiring stoma bags where the stool comes out from the abdomen. Furthermore, Gastrointestinal (GI) bleeding is often associated with peptic ulcer disease (PUD) and GI perforations and can be fatal if not treated immediately. Suspected bleeding PUD patients can be diagnosed and treated endoscopically in an emergency room, an intensive care unit (ICU) or a GI suite. Surgery generally results in higher costs, morbidity and mortality than endoscopy, thus neither laparoscopy nor invasive surgery are preferred, unless there is no endoscopic alternative or endoscopy efforts have failed. If the diseased tissue is beyond repair, a surgical gastric resection may be performed.

At present, the two most commonly used endoscopic treatments for preventing GI bleeding are thermal therapy and injection therapy. Less common techniques include endoclipping, laser cautery and argon plasma cautery. With thermal therapy, a catheter with a rigid heating element tip is passed through the working channel of an endoscope after the bleeding site is visualized and diagnosed. After the rigid catheter tip has exited the scope, the scope is manipulated to press the tip against the bleed site. Thermal power is applied, either through a resistive element in the tip or by applying RF energy through the tissue, thus desiccating and cauterizing the tissue. The combination of the tip compressing the tissue/vessel and the application of heat theoretically welds the vessel closed.

Although thermal treatment is fairly successful in achieving hemostasis, it often takes more than one attempt and there is frequent re-bleeding. Generally, several pulses of energy are applied during each attempt. If early re-treatment is needed, there is a risk of perforation with the heat probe.

With injection therapy, a catheter with a distally extendable hypodermic needle is passed through the working channel of the endoscope after the bleeding has been visualized and diagnosed. Once the catheter tip has exited the scope, the scope is manipulated to the bleed site, the needle is extended remotely and inserted into the bleed site. A vasoconstricting (narrowing of blood vessels) or sclerosing (causing a hardening of tissue) drug is then injected through the needle. Multiple injections in and around the bleeding site are often needed, until hemostasis has been achieved. As with thermal therapy, re-bleeding can also be a problem.

An endoclip is a metallic mechanical device used in endoscopy in order to close two mucosal surfaces without the need for surgery and suturing. Its function is similar to a suture in gross surgical applications, as it is used to join together two disjointed surfaces, but the endoclip can be applied through the channel of an endoscope under direct visualization. Endoclips have found use in treating gastrointestinal bleeding (both in the upper and lower GI tract), in preventing bleeding after therapeutic procedures such as polypectomy, and in closing gastrointestinal perforations.

Numerous types of bleeding lesions have been successfully clipped with endoclips, including bleeding peptic ulcers, Mallory-Weiss tears of the esophagus, Dieulafoy's lesions, stomach tumors and bleeding after removal of polyps. Bleeding peptic ulcers require endoscopic treatment if they show evidence of high-risk stigmata of re-bleeding, such as evidence of active bleeding or oozing on endoscopy or the presence of a visible blood vessel around the ulcer.

Despite widespread usage, endoclips suffer the disadvantage of remaining permanently closed, i.e., once jaw closure begins, it is not possible to reopen the jaws of the endoclip. If the endoscopist only triggered the jaws to close when the endoclip was perfectly positioned, this would not be an issue. However, because the vessel is frequently difficult to see, several clips must typically be deployed to successfully pinch the vessel to achieve hemostasis.

Expandable mesh occluders have been used for a wide variety of different endoscopic treatments, ranging from sealing cardiac defects to occluding sinus cavities. A common example is the usage of an expanding mesh occluder for the repair of septal defects, where the expanding mesh occluder is carried in a collapsed state on a guidewire of an endoscopically steered catheter. Once the site of the defect has been reached, the guidewire is deployed and the occluder expands. Given both the ease of use and the high success rate of such endoscopic occluder procedures, particularly with septal defect occluders, it would be desirable to be able to adapt the procedure for the repair of gastrointestinal defects. Thus, an endoscopic method of sealing a gastrointestinal defect solving the aforementioned problems is desired.

### SUMMARY

The endoscopic method and device for sealing a gastrointestinal defect is provided to treat gastrointestinal defect leaks in a more minimally invasive and efficient way. What is proposed is a device that fits through a standard gastroscope or colonoscope with a deployable defect closure system on the end. The deployment will first involve advancing a built-in guidewire through the defect and then releasing a plug over the defect. After which, through the use of the endoscope, gastroscope, or colonoscope, the plug will be secured in place and thus prevent any further leakages from the gastrointestinal defect. In certain embodiments, the plug can be a dumb-bell shaped plug. This will not only help an immediate closure of these defects but will also be minimally invasive to the patient, as this does not require an actual surgical operation. The device is most applicable to focusing on treatment of stomach leaks, as these are the most commonly seen complication around the world following laparoscopic sleeve gastrectomy and gastric bypass operations.

These and other features of the present subject matter will become readily apparent upon further review of the following specification.

### BRIEF DESCRIPTION OF THE DRAWINGS

FIG. 1 is a perspective view of an endoscopic method for sealing a gastrointestinal defect as shown in four stages from the topmost diagram to the bottommost diagram.

FIG. 2 is top side view in section of an endoscopic device for sealing a gastrointestinal defect.

FIG. 3 is a perspective view of the endoscopic device for sealing a gastrointestinal defect.

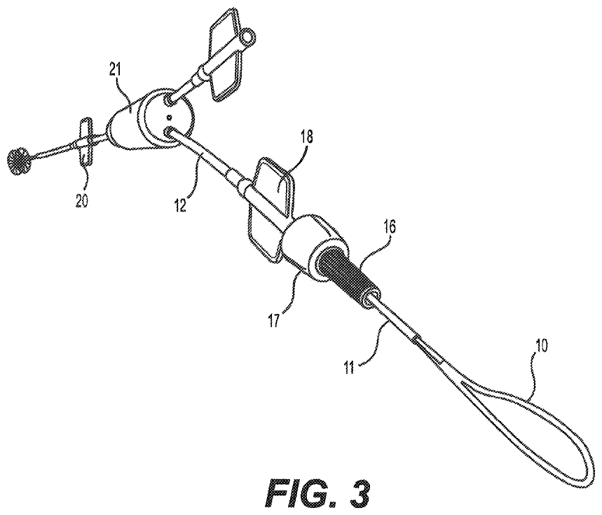

FIG. 4 is a side view in section of the endoscopic device for sealing a gastrointestinal defect.

FIG. 5 is a perspective view of a subject having a gastrointestinal defect.

FIG. 6 is a side view of the endoscopic device in approaching a gastrointestinal defect.

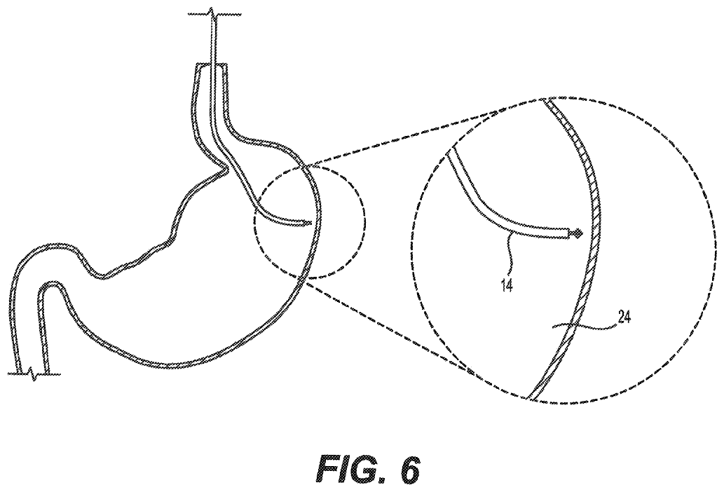

FIG. 7 is a perspective view of the endoscopic device for sealing a gastrointestinal defect.

FIG. 8 is a cross section of a distal end of the endoscopic device.

FIG. 9 is a side view of the distal end of the endoscopic device showing a configuration of the flattened or collapsed gastrointestinal mesh plug being extended into position.

FIG. 10 is a side view of the distal end of the endoscopic device showing a configuration of the flattened or collapsed gastrointestinal mesh plug being retracted prior to being extended into position.

Similar reference characters denote corresponding features consistently throughout the attached drawings.

### DETAILED DESCRIPTION OF EMBODIMENTS

The endoscopic method and device for sealing a gastrointestinal defect is provided herewith mainly as a complement to a standard endoscope, gastroscope, or colonoscope in that it is adaptable to the particular configuration of the scope and no scope needs to be specially modified to practice the method and the device that accompanies the method.

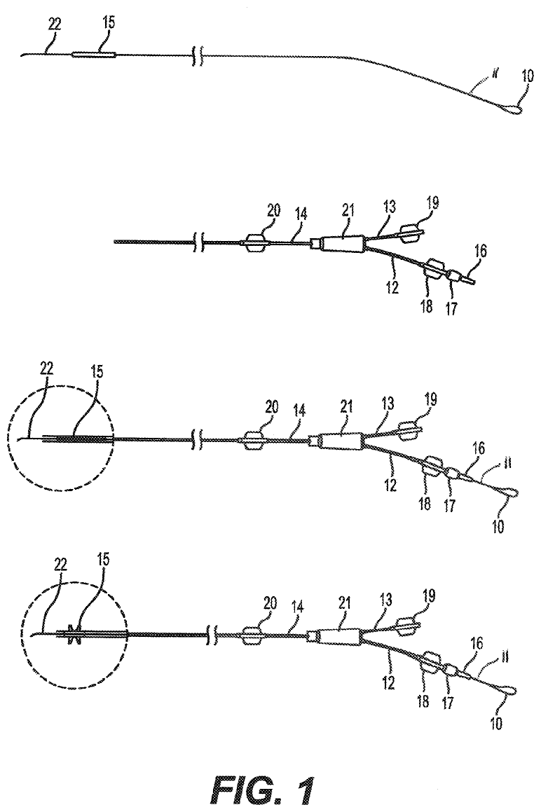

FIG. 1 depicts the method in that attaches to a standard scope (not shown). In the embodiment of FIG. 1, a top most deployment hub (13 & 19) provides the gastrointestinal defect plug (15) and bottom most deployment hub (12 & 18) provides the guidewire (11) which helps to secure the plug in place. Both deployment hubs can drive the gastrointestinal defect plug (15) and the guidewire (11) forward towards the area of the gastrointestinal hub. The topmost diagram of FIG. 1 is a depiction of the guidewire (11) by itself with the flattened or collapsed gastrointestinal plug (15). The second from the topmost diagram through the bottommost diagram of FIG. 1 depict an insertion motion of both the guidewire (11) and the flattened or collapsed gastrointestinal plug (15) into the area of the gastrointestinal leak as shown in the dotted circles in the diagrams, respectively.

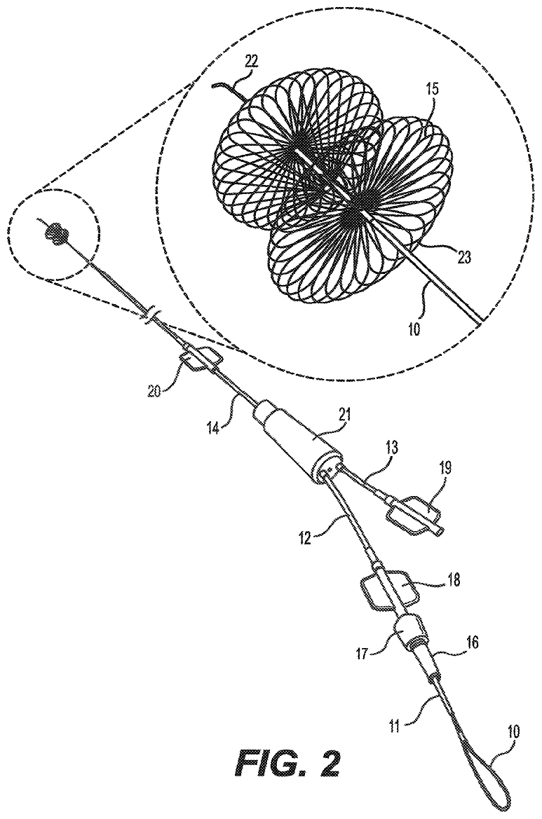

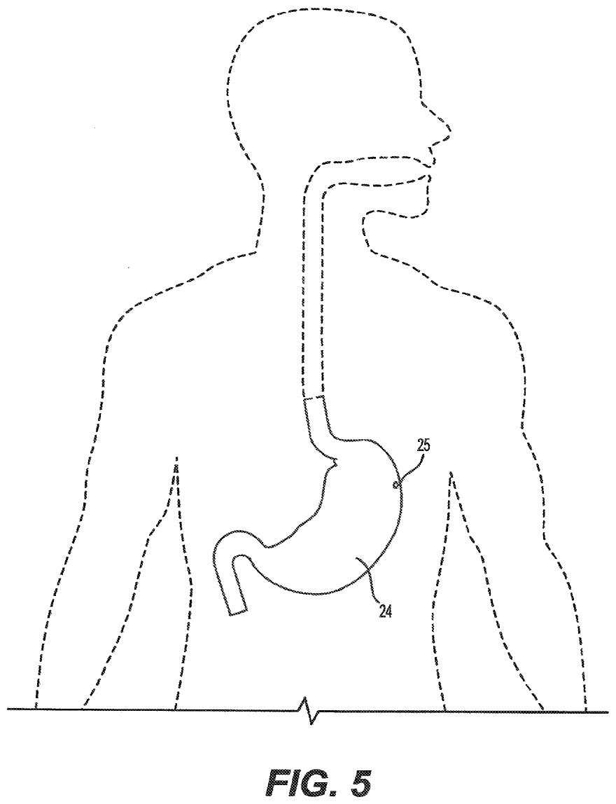

In the detailed view provided by FIG. 2, a guidewire tip protector (10) acts as a sheath for the guidewire (11) which slides through the sheath and by means of guidewire deployment hub components (12 & 18). The guidewire tip protector (10) shields the guidewire (11) as it navigates to the area of the defect. Furthermore, a protective sleeve (14) and stabilizing device (20) is provided for protection of the gastrointestinal defect plug as it is manipulated closer to the defect area (25) as shown in FIG. 5.

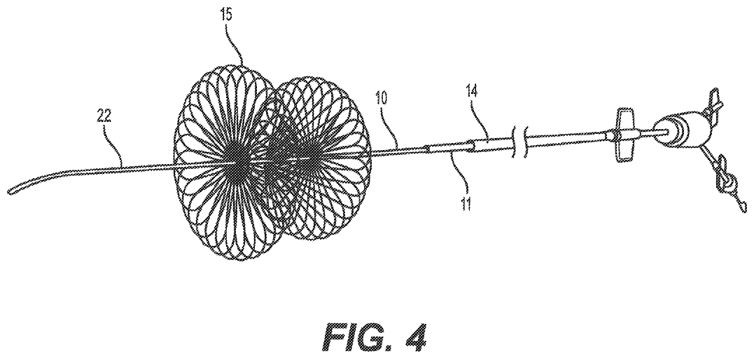

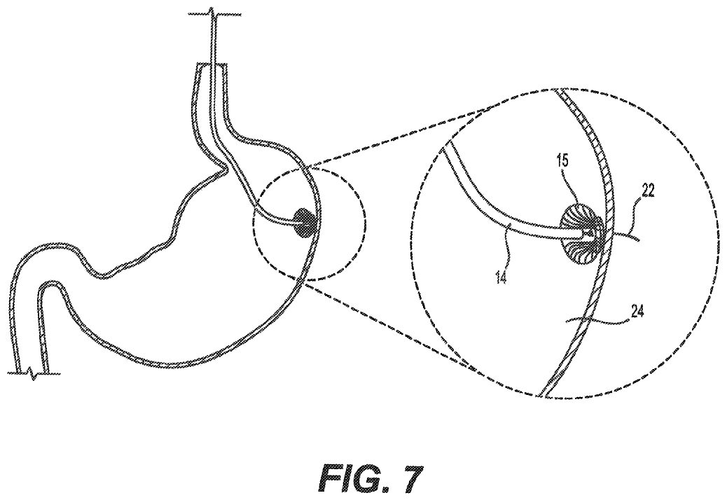

In certain embodiments, the flattened or collapsed gastrointestinal plug is constructed from a mesh made of Teflon™ and is constructed much in the manner of meshes used to close heart defects. In the treating of a gastrointestinal defect, the mesh plug (15) is flattened or collapsed around the guidewire (11) when inserted into the defect area as shown in FIG. 1A-1. The flattened or collapsed mesh plug (15) is positioned just short of the terminal point of the distal end (22) such that when the distal end (22) is extended through the gastrointestinal defect (24), a first segment of the flattened or collapsed mesh plug (15) remains within the interior abdominal wall and a second segment of the flattened or collapsed mesh plug (15) protrudes from the gastrointestinal defect (25) past the outer surface of the abdominal wall as shown in FIG. 7. Once the flattened or collapsed mesh plug (15) is positioned where desired by the guidewire (11), it is expanded to seal the gastrointestinal defect (24) by having the first lobe expanded and positioned on the outer surface of the abdominal wall and having the second lobe expanded and positioned on the inner surface of the abdominal wall as shown in FIGS. 4 and 7. In FIG. 7, only the lobe on the interior surface of the abdominal wall is shown as expanded. Not shown in FIG. 7 is the second lobe on the exterior surface of the abdominal wall. However, when fully deployed, the mesh plug (15) extends out in a dual lobe configuration as shown in FIG. 4. Once the deployed and expanded gastrointestinal plug is in place, the distal end of the guidewire (22) is retracted back into the sheath (10) and the deployed and expanded gastrointestinal plug is detached from the guidewire (11) and left in place. In certain embodiments, a biosafe nylon thread can be left connected to the plug after deployment so that the deployed and expanded gastrointestinal plug (15) can be repositioned if need be by standard endoscopic graspers, as well as facilitating a quick removal of the plug when required due to migration. In this regard, by way of non-limiting example, the nylon thread can be about 5 cm long.

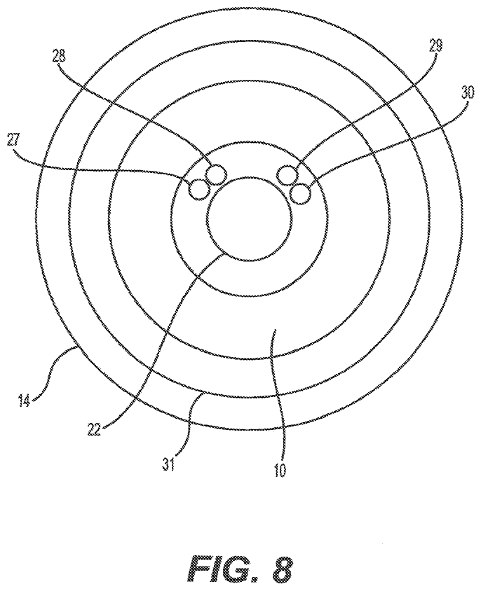

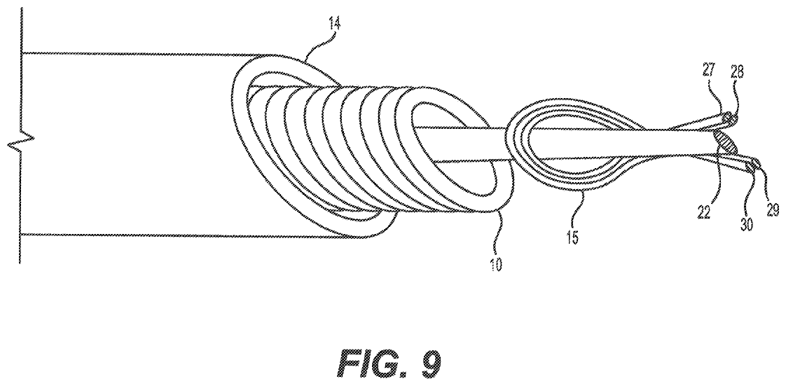

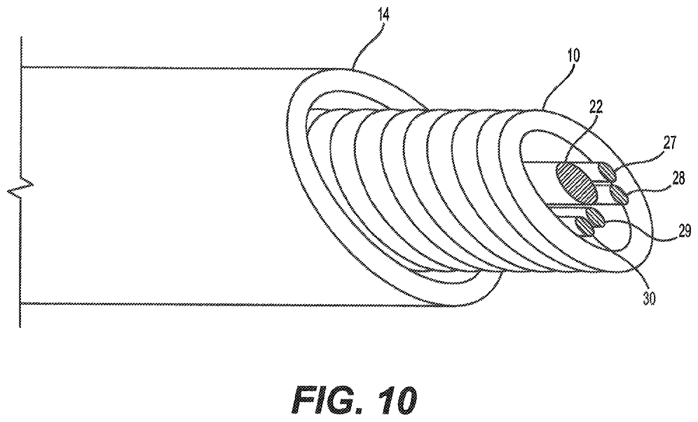

FIG. 8 depicts a cross section of the flattened or collapsed gastrointestinal mesh plug (15) and guidewire (11) configuration of the device proximal to the distal end of the guidewire (22). The cross section depicts a series of concentric circles which show cross sections of the distal end of the guide wire (22), guidewire tip protector (10), the device protective sleeve (14), and a gap (31) between the guidewire tip protector (10) and the device protective sleeve (14). In FIGS. 9 and 10, which depict a side view of the flattened or collapsed gastrointestinal mesh plug (15) and guidewire (11) configuration, the flattened or collapsed gastrointestinal mesh plug (15) is shown as being wrapped proximal to the distal end of the guidewire (22) by means of terminating limbs (27, 28, 29, 30) of the flattened or collapsed gastrointestinal mesh plug (15). FIG. 10 depicts a completely retracted arrangement of the flattened or collapsed gastrointestinal mesh plug (15) and guidewire (11) configuration prior to being fully deployed. FIG. 9 depicts an extension operation of the flattened or collapsed gastrointestinal mesh plug (15) and guidewire (11) of the configuration into the area of the defect prior to be being expanded into the dual lobe configuration as discussed above (FIG. 2, element 15).

It is to be understood that the endoscopic method and device for sealing a gastrointestinal defect is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An endoscopic method for sealing a gastrointestinal defect, comprising:
positioning a distal end of a guidewire of an endoscope near an area of said gastrointestinal defect;
positioning a gastrointestinal plug proximally located on said guidewire and near said distal end within an opening of the gastrointestinal defect;
expanding the gastrointestinal plug to an expanded configuration which expands a first segment of said gastrointestinal plug along an interior wall of said gastrointestinal defect to obtain an expanded first segment and which expands a second segment of said gastrointestinal plug along an outer wall of said gastrointestinal defect to obtain an expanded second segment; and
securing the expanded first segment and the expanded second segment of the gastrointestinal plug in place to seal off said gastrointestinal defect.

2. The endoscopic method for sealing a gastrointestinal defect as recited in claim 1, wherein said expanded configuration is a dual lobe configuration.

3. The endoscopic method for sealing a gastrointestinal defect as recited in claim 2, wherein the gastrointestinal plug is a mesh.

4. The endoscopic method for sealing a gastrointestinal defect as recited in claim 3, wherein the gastrointestinal plug is a mesh made from polytetrafluoroethylene.

5. The endoscopic method for sealing a gastrointestinal defect as recited in claim 4, wherein a thread is left attached to the expanded first segment of the gastrointestinal plug after securing the expanded first segment and the expanded second segment of the gastrointestinal plug in place to seal off said gastrointestinal defect.

6. The endoscopic method for sealing a gastrointestinal defect as recited in claim 1, wherein said gastrointestinal defect is selected from one or more of the group consisting of an ulcer, a gastrointestinal perforation, a post-bariatric surgery leak, and a gastrointestinal fistula.

7. A device for use with an endoscope for sealing a gastrointestinal defect, comprising:
a first deployment hub for positioning a distal end of a guidewire of an endoscope near an area of said gastrointestinal defect;
a second deployment hub for positioning a gastrointestinal plug proximally located on said guidewire and near said distal end within an opening of the gastrointestinal defect, said second deployment hub also configured for expanding the gastrointestinal plug to an expanded configuration including an expanded first segment of said gastrointestinal plug along an interior wall of said gastrointestinal defect to obtain and an expanded second segment of said gastrointestinal plug along an outer wall of said gastrointestinal of said gastrointestinal defect; and
said first deployment hub being configured for securing the expanded first segment and the expanded second segment of the gastrointestinal plug in place to seal off said gastrointestinal defect.

8. The device for use with an endoscope for sealing a gastrointestinal defect as recited in claim 7, wherein said expanded configuration is a dual lobe configuration.

9. The device for use with an endoscope for sealing a gastrointestinal defect as recited in claim 8, wherein the gastrointestinal plug is a mesh.

10. The device for use with an endoscope for sealing a gastrointestinal defect as recited in claim 9, wherein the gastrointestinal plug is a mesh made from polytetrafluoroethylene.

11. The device for use with an endoscope for sealing a gastrointestinal defect as recited in claim 10, wherein a thread is left attached to the expanded first segment of the gastrointestinal plug after securing the expanded first segment and the expanded second segment of the gastrointestinal plug in place to seal off said gastrointestinal defect.
